(19) United States
(12) Patent Application Publication
(10) Pub. No.: US 2021/0177272 A1
(43) Pub. Date: Jun. 17, 2021

(54) Living Body Internal Temperature Measuring Device and Living Body Internal Temperature Measuring Method

(71) Applicant: Nippon Telegraph and Telephone Corporation, Tokyo (JP)

(72) Inventors: Michiko Seyama, Tokyo (JP); Daichi Matsunaga, Tokyo (JP); Yujiro Tanaka, Tokyo (JP)

(21) Appl. No.: 17/059,031

(22) PCT Filed: May 15, 2019
(86) PCT No.: PCT/JP2019/019253

§ 371 Date: Nov. 25, 2020

(30) Foreign Application Priority Data
Jun. 1, 2018 (JP) 2018-105894

(51) Int. Cl. A61B 5/01; A61B 5/026; A61B 5/00

(52) U.S. Cl. CPC A61B 5/01; A61B 2562/0271; A61B 5/6846; A61B 5/026

(57) ABSTRACT

An internal body temperature measurement device includes: a temperature sensor which measures an epidermis temperature of a living body; a heat flux sensor which measures a magnitude of a heat flux discharged from a body surface of the living body; a blood flow sensor which measures a blood flow rate in a vicinity of the heat flux sensor; and a storage unit which stores a relation between the blood flow rate in the vicinity of the heat flux sensor and a core temperature depth. A correction amount of the core temperature depth corresponding to the blood flow rate in the vicinity of the heat flux sensor is obtained on a basis of the relation stored in the storage unit, and a core temperature of the living body is calculated from the epidermis temperature, the magnitude of the heat flux, and the correction amount of the core temperature depth.

### CROSS-REFERENCE TO RELATED APPLICATIONS

This application is a national phase entry of PCT Application No. PCT/JP2019/019253, filed on May 15, 2019, which claims priority to Japanese Application No. 2018-105894, filed on Jun. 1, 2018, which applications are hereby incorporated herein by reference.

### TECHNICAL FIELD

The present invention relates to an internal body temperature measurement device and an internal body temperature measurement method for measuring a temperature of a core part of a living body.

### BACKGROUND

A living body has a temperature region which is not affected by a change in an outside air temperature or the like beyond a certain depth from an epidermis toward a core part (see FIG. 11) (hereinafter, the temperature of the region is referred to as a “core temperature” or a “deep body temperature”). It is known that measuring a fluctuation of the core temperature is useful for grasping an internal body rhythm.

In order to measure the core temperature, a percutaneous temperature measurement method rather than an invasive measurement such as indwelling is easy and useful for routine body temperature management.

### CITATION LIST

### Non-Patent Literature

Non-Patent Literature 1: Akio Nakayama, “Handbook of physiological sciences: Volume 22”, Igaku-Shoin (1987);
Non-Patent Literature 2: Shinya Nakagawa et al., “Wearable Core Temperature Thermometer Implemented by the MEMS Heat Flux Sensor”, IEEJ Transactions on Electronics, Vol. 135 (2015) No. 8, p. 343-348.

### SUMMARY

### Technical Problem

However, in a conventional percutaneous body temperature measurement device, it is difficult to accurately measure the core temperature. One of the reasons for that is that the apparent depth from the epidermis to the temperature region of the core temperature is changed due to a blood flow, and the measurement value is changed.

Generally, it is known that the depth from the epidermis to the temperature region of the core temperature Tc (hereinafter, referred to as a “core temperature depth” in some cases) depends on a blood flow rate as illustrated in FIG. 12 (Non-Patent Literature 1, FIG. 59). The blood flow of a body surface increases when a blood vessel presents in a dermis layer and called arteriovenous anastomoses (AVA) is dilated by a neural activity of the body. When the blood flow rate of the body surface increases, the heat energy of the core part moves together with the blood flow to a surface layer, and thus the apparent depth from the epidermis to the temperature region of the core temperature Tc becomes shallow.

On the other hand, in order to measure the core temperature Tc of a living body 90 in a percutaneous manner, for example, a heat flux sensor 20 illustrated in FIG. 13 is used to measure a temperature Tu of an upper surface of a heat resistor 20r configuring the heat flux sensor 20 and a temperature of a lower surface, that is, an epidermis temperature Ts, and the core temperature Tc is calculated from a value Rx of the heat resistance of the subcutaneous tissue of the living body 90 and a vertical heat resistance value Rr of the heat resistor 20r on the basis of following Formula (1) (Non-Patent Literature 2).

$$\begin{matrix}[ Formula 1 ] & \\ T c = T s + \frac{R x}{R r} ( Ts - Tu ) & ( 1 )\end{matrix}$$

In order to measure the core temperature Tc of the living body 90 in the percutaneous manner, the value Rx of the heat resistance of the subcutaneous tissue of the living body 90 is required. However, when the core temperature depth is changed along with the change in the blood flow rate of the body surface, the value Rx of the heat resistance of the subcutaneous tissue of the living body 90 is also changed, and thus, it is difficult to accurately measure the core temperature Tc.

In this regard, an object of embodiments of the present invention is to provide an internal body temperature measurement device capable of more accurately measuring a core temperature with a percutaneous temperature measurement method.

### Means for Solving the Problem

In order to achieve the above-described object, an internal body temperature measurement device according to embodiments of the present invention includes: a temperature sensor (20S) which measures an epidermis temperature of a living body; a heat flux sensor (20) which measures a magnitude of a heat flux discharged from a body surface of the living body; a blood flow sensor (30) which measures a blood flow rate in a vicinity of the heat flux sensor; a storage unit (50) which stores a relation between the blood flow rate in the vicinity of the heat flux sensor and a parameter regarding a core temperature of the living body; and an arithmetic circuit (40) configured to obtain a correction amount of a value of the parameter corresponding to the blood flow rate in the vicinity of the heat flux sensor on a basis of the relation stored in the storage unit and calculate the core temperature of the living body from the epidermis temperature measured by the temperature sensor, the magnitude of the heat flux measured by the heat flux sensor, and the correction amount of the value of the parameter.

In the internal body temperature measurement device according to embodiments of the present invention, the arithmetic circuit (40) may include a first calculation unit (41) which calculates the correction amount of the value of the parameter corresponding to the blood flow rate in the vicinity of the heat flux sensor on the basis of the relation stored in the storage unit, and a second calculation unit (42) which calculates the core temperature of the living body on a basis of the epidermis temperature measured by the temperature sensor, the magnitude of the heat flux measured by the heat flux sensor, and the correction amount of the value of the parameter calculated by the first calculation unit.

As one configuration example of the internal body temperature measurement device according to embodiments of the present invention, the second calculation unit (42) may include an estimation unit (421) which estimates a heat resistance between an epidermis and a core part of the living body from the magnitude of the heat flux measured by the heat flux sensor, a correction unit (422) which corrects the heat resistance estimated by the estimation unit on a basis of the correction amount of the value of the parameter, and a core temperature calculation unit (423) which calculates the core temperature of the living body from the epidermis temperature measured by the temperature sensor, the magnitude of the heat flux measured by the heat flux sensor, and the heat resistance obtained by the correction of the correction unit.

As one configuration example of the internal body temperature measurement device according to embodiments of the present invention, the second calculation unit may include an estimation unit which estimates a depth (core temperature depth) from an epidermis to a core part of the living body from the magnitude of the heat flux measured by the heat flux sensor, a correction unit which corrects the depth from the epidermis to the core part of the living body estimated by the estimation unit on a basis of the correction amount of the value of the parameter, and a core temperature calculation unit which calculates the core temperature of the living body from the epidermis temperature measured by the temperature sensor, the magnitude of the heat flux measured by the heat flux sensor, and the depth from the epidermis to the core part of the living body obtained by the correction of the correction unit.

As one configuration example of the internal body temperature measurement device according to embodiments of the present invention, the second calculation unit (42a) may include an estimation unit (421a) which estimates the core temperature of the living body from the epidermis temperature measured by the temperature sensor and the magnitude of the heat flux measured by the heat flux sensor, and a core temperature calculation unit (423a) which calculates the core temperature of the living body by correcting the core temperature of the living body estimated by the estimation unit on a basis of the correction amount of the value of the parameter calculated by the first calculation unit.

In the internal body temperature measurement device according to embodiments of the present invention, the parameter may be the depth from the epidermis to the core part of the living body, the heat resistance, or the core temperature.

The internal body temperature measurement device according to embodiments of the present invention further may include at least two of the blood flow sensors. The arithmetic circuit may obtain a representative value of respective blood flow rates measured by the at least two blood flow sensors and obtain the correction amount of the value of the parameter corresponding to the blood flow rate in the vicinity of the heat flux sensor on a basis of the representative value of the blood flow rates and the relation stored in the storage unit.

An internal body temperature measurement method according to embodiments of the present invention includes: a step of measuring an epidermis temperature of a living body and a magnitude of a heat flux discharged from a body surface of the living body; a step of measuring a blood flow rate of the body surface; and a step of obtaining, on a basis of a previously prepared relation between a blood flow rate in a vicinity of a heat flux sensor and a parameter regarding a core temperature of the living body, a correction amount of a value of the parameter corresponding to the blood flow rate in the vicinity of the heat flux sensor, and calculating the core temperature of the living body from the epidermis temperature measured by the temperature sensor, the magnitude of the heat flux measured by the heat flux sensor, and the correction amount of the value of the parameter.

### Effects of Embodiments of the Invention

According to embodiments of the present invention, the value of the parameter regarding the core temperature of the living body is corrected according to the blood flow rate of the body surface, and thus the core temperature can be measured more accurately with the percutaneous temperature measurement method.

### BRIEF DESCRIPTION OF THE DRAWINGS

FIG. 1 is a diagram illustrating a configuration of an internal body temperature measurement device according to a first embodiment of the present invention.

FIG. 2 is a diagram illustrating the configuration of the internal body temperature measurement device according to the first embodiment of the present invention.

FIG. 3 is a diagram for explaining a positional relation between a heat flux sensor and a blood flow sensor in the internal body temperature measurement device according to the first embodiment.

FIG. 4 is a diagram illustrating a relation between a blood flow rate and a depth from an epidermis to a temperature region of a core temperature.

FIG. 5 is a flowchart for explaining an operation of the internal body temperature measurement device according to the first embodiment.

FIG. 6 is a diagram illustrating one example of a measurement result by the internal body temperature measurement device according to the first embodiment.

FIG. 7 is a diagram illustrating a configuration of an internal body temperature measurement device according to a second embodiment of the present invention.

FIG. 8 is a diagram for explaining a positional relation between the heat flux sensor and the blood flow sensor.

FIG. 9 is a diagram for explaining a positional relation between the heat flux sensor and the blood flow sensor.

FIG. 10 is a flowchart for explaining an operation of the internal body temperature measurement device according to the second embodiment.

FIG. 11 is a diagram for schematically explaining a temperature distribution of a subcutaneous tissue of a living body.

FIG. 12 is a diagram illustrating a relation between the depth from the epidermis to the temperature region of the core temperature and the blood flow rate.

FIG. 13 is a schematic diagram for explaining the measurement of the core temperature by the heat flux sensor including a temperature sensor

### DETAILED DESCRIPTION OF ILLUSTRATIVE EMBODIMENTS

Hereinafter, embodiments of the present invention will be described with reference to the drawings.

### First Embodiment

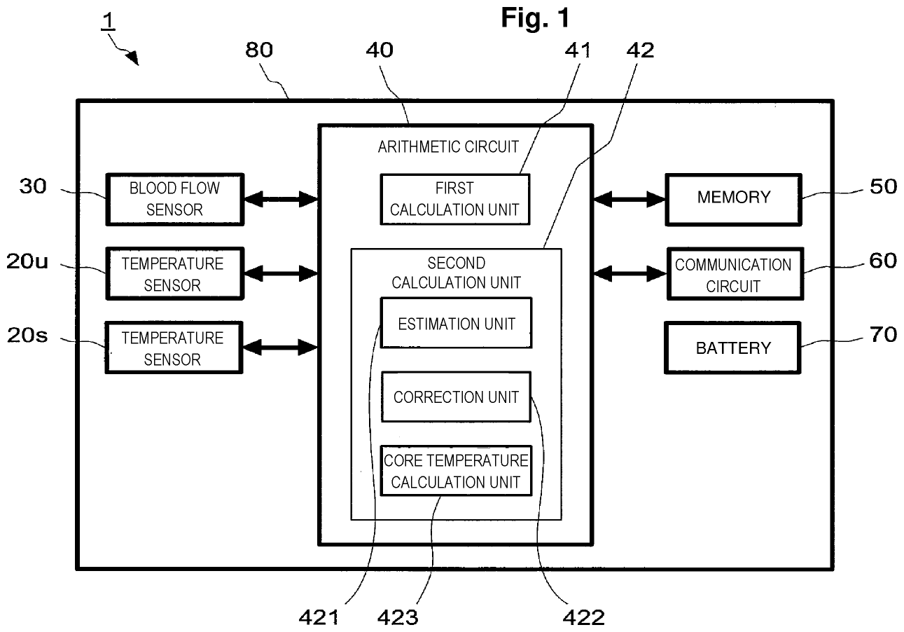

As illustrated in FIG. 1, an internal body temperature measurement device 1 according to a first embodiment of the present invention includes, on a sheet-shaped base material 80, a heat flux sensor 20, a blood flow sensor 30, an arithmetic circuit 40, a memory 50 which functions as a storage unit, a communication circuit 6o which functions as an I/F circuit with an outside, and a battery 70 which supplies power to the arithmetic circuit 40, the communication circuit 60, or the like.

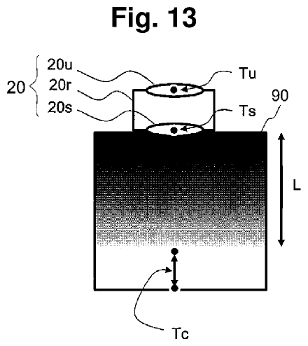

Herein, the heat flux sensor 20 is a device which measures the movement of heat per unit time/unit area. As illustrated in FIG. 13, in this embodiment, the heat flux sensor 20 having a temperature sensor 20U and a temperature sensor 20S is used for the upper surface and the lower surface of the heat resistor tor, respectively. The magnitude of heat flux discharged from the body surface of the living body 90 is measured, and at the same time, an epidermis temperature Ts is measured by the temperature sensor 20S. As the temperature sensors 20U and 20S, a well-known thermistor, a thermopile using a thermocouple, an ultrasonic thermometer utilizing the change in sound velocity according to the temperature, an infrared temperature sensor or another optical thermometer utilizing the change in a light absorption rate according to the temperature, or the like is used, for example.

The blood flow sensor 30 is a device which is arranged near the heat flux sensor 20 and measures the blood flow rate of the body surface of the living body 90. As such a blood flow sensor 30, a laser Doppler blood flowmeter or another optical blood flow sensor which measures a blood flow rate in a subcutaneous tissue by irradiating a skin with a laser or an ultrasonic blood flowmeter is used, for example.

The memory 50 stores a relation between the blood flow rate in the vicinity of the heat flux sensor 20, that is, the body surface and parameters regarding the core temperature Tc of the living body. Herein, the parameters regarding the core temperature Tc of the living body are, for example, a depth (core temperature depth) L from the epidermis to the core part in the living body, a heat resistance Rx of the subcutaneous tissue between the epidermis and the core part in the living body, or the core temperature Tc. The relation between the blood flow rate of the body surface and the parameters regarding the core temperature Tc of the living body may be stored in the memory 50 in the form of a table. However, the relation also may be stored as a function.

Further, the memory 50 stores the time-series data of the core temperature which is estimated and calculated from the result obtained by the measurement of the heat flux sensor 20, that is, the time-series data in which the core temperature and the time when the core temperature is measured are associated with each other.

The arithmetic circuit 40 obtains the correction amount of the value of the parameter corresponding to the blood flow rate in the vicinity of the heat flux sensor 20 on the basis of the relation between the blood flow rate of the body surface stored in the memory 50 and the parameter regarding the core temperature Tc of the living body. Further, the arithmetic circuit 40 calculates the core temperature Tc of the living body from the epidermis temperature Ts measured by the temperature sensor 20S, the magnitude of the heat flux measured by the heat flux sensor 20, and the correction amount of the value of the parameter.

Such an arithmetic circuit 40 can be formed from an arithmetic device and a computer program. For example, the arithmetic circuit 40 can be formed from a first calculation unit 41 which calculates the correction amount of the value of the parameter corresponding to the blood flow rate in the vicinity of the heat flux sensor 20 on the basis of the relation between the blood flow rate and the parameter stored in the memory 50 and a second calculation unit 42 which calculates the core temperature of the living body on the basis of the epidermis temperature Ts measured by the temperature sensor 20S, the magnitude of the heat flux measured by the heat flux sensor 20, and the correction amount of the value of the parameter calculated by the first calculation unit 41.

In the internal body temperature measurement device 1 according to this embodiment, for example, in a case where the heat resistance Rx between the epidermis and the core part in the living body is adopted as the parameter, the first calculation unit 41 is configured to calculate the correction amount ΔR of the heat resistance Rx between the epidermis and the core part of the living body corresponding to the blood flow rate in the vicinity of the heat flux sensor 20. Further, the second calculation unit 42 is formed from an estimation unit 421 which estimates the heat resistance Rx between the epidermis and the core part of the living body from the magnitude of the heat flux measured by the heat flux sensor 20, a correction unit 422 which corrects the heat resistance Rx estimated by the estimation unit 421 on the basis of the correction amount ΔR of the heat resistance calculated by the first calculation unit 41, and a core temperature calculation unit 423 which calculates the core temperature Tc of the living body from the epidermis temperature Ts measured by the temperature sensor 20S, the magnitude of the heat flux measured by the heat flux sensor 20, and the heat resistance Rx+ΔR obtained by the correction of the correction unit 422.

In a case where the core temperature depth L is adopted as the parameter, the first calculation unit 41 is configured to calculate the correction amount ΔL of the core temperature depth L corresponding to the blood flow rate in the vicinity of the heat flux sensor 20. Further, the estimation unit 421 may be configured to estimate the depth (core temperature depth) L from the epidermis to the core part of the living body from the magnitude of the heat flux measured by the heat flux sensor 20. The correction unit 422 may be configured to correct the core temperature depth L of the living body estimated by the estimation unit 421 on the basis of the correction amount ΔL of the core temperature depth L calculated by the first calculation unit 41. The core temperature calculation unit 423 may be configured to calculate the core temperature Tc of the living body from the epidermis temperature Ts measured by the temperature sensor 20S, the magnitude of the heat flux measured by the heat flux sensor 20, and the depth L+ΔL from the epidermis to the core part of the living body obtained by the correction by the correction unit 422.

The communication circuit 6o is an I/F circuit which outputs the time-series data of the temperature obtained by the correction of the arithmetic circuit 40 to the outside or outputs an alarm when an error occurs. As such a communication circuit 60, an output circuit to which a USB or other cable can be connected is used in a case where data or the like is output by wire. For example, a wireless communication circuit based on Bluetooth (registered trademark) or the like may be used.

The sheet-shaped base material 8o does not only function as a base for placing the heat flux sensor 20, the blood flow sensor 30, the arithmetic circuit 40, the memory 50, the communication circuit 60, and the battery 70 but also includes wiring (not illustrated) which connects those elements electrically. Considering that the internal body temperature measurement device 1 is placed on the epidermis of the living body, a deformable flexible substrate is desirably used as the sheet-shaped base material 80.

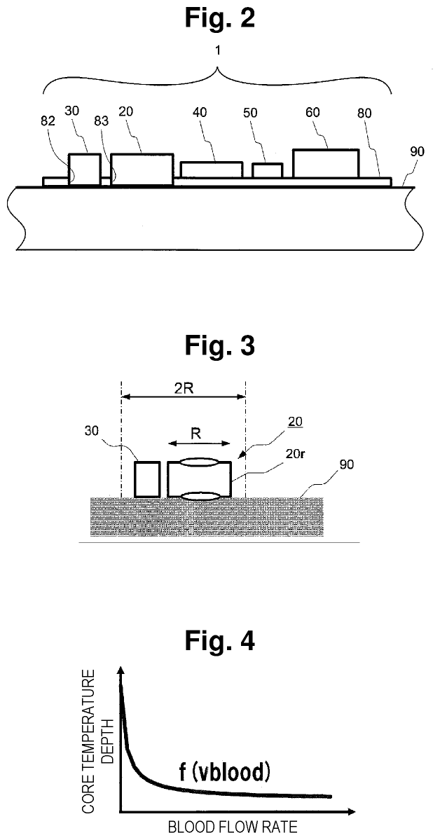

Further, as illustrated in FIG. 2, openings 82 and 83 are provided in a part of the sheet-shaped base material 80, and the heat flux sensor 20 and the blood flow sensor 30 are placed on the base material 80 so as to be in contact with the epidermis of the living body through the openings 82 and 83.

If the heat resistor 20r of the heat flux sensor 20 is formed, for example, in a disc shape, a region of about twice a diameter R of the heat resistor 20r affects the measurement of the core temperature Tc. Therefore, in order to measure the blood flow rate of the body surface in the vicinity of the heat flux sensor 20 with the blood flow sensor 30, as illustrated in FIG. 3, the blood flow sensor 30 is installed within the region of twice the diameter R of the heat resistor 20r of the heat flux sensor 20, that is, about a diameter 2R around the heat resistor 20r in plan view. One or multiple blood flow sensors 30 can be also provided in one heat flux sensor 20, but in this embodiment, one blood flow sensor 30 is provided in one heat flux sensor 20.

[Measurement Principle of Internal Body Temperature Measurement Device]

Next, the measurement principle of the internal body temperature measurement device according to this embodiment will be described.

As illustrated in FIG. 13, the living body 90 has the region of the temperature which is not affected by a change in an outside air temperature or the like beyond a certain depth from the epidermis in a depth direction of the subcutaneous tissue, that is, the core temperature. Typically, the epidermis temperature Ts is lower than the core temperature Tc, and a temperature gradient is generated from the core part toward the epidermis.

As described above with Formula (1), in order to measure the core temperature Tc of the living body 90 in a percutaneous manner, the value Rx of the heat resistance of the subcutaneous tissue is required.

On the other hand, the heat resistance Rx of the subcutaneous tissue is proportional to the depth (core temperature depth) L from the epidermis to the temperature region of the core temperature Tc of the core part and is inversely proportional to a thermal conductivity k of the subcutaneous tissue.

Rx=L/k(Tc−Ts) (2)

The thermal conductivity k is determined by a subcutaneous composition. However, the subcutaneous composition does not change in the short term, and thus the heat resistance Rx of the subcutaneous tissue depends on a distance L from the epidermis to the temperature region of the core temperature Tc. As described above, the apparent core temperature depth changes along with the blood flow rate of the body surface (FIG. 12), but in the related art, the change in the depth to the temperature region of the core temperature Tc is not taken into consideration.

In this regard, in this embodiment, the depth (core temperature depth) L from the epidermis to the core part of the living body is adopted as the parameter, and the relation L=f(vblood) between the blood flow rate vblood and the apparent core temperature depth L is stored in advance in the memory 50 as illustrated in FIG. 4. At this time, the change amount ΔR of the heat resistance of the subcutaneous tissue generated by a change amount Δvblood of the blood flow rate can be calculated from the change amount ΔL of the depth obtained from the relation L=f(vblood) and the change amount Δvblood of the blood flow rate, and the thermal conductivity k of the subcutaneous tissue on the basis of the following formula.

ΔL=Δvblood×f(vblood) (3)

ΔR=ΔL/k (4)

The correction amount ΔTc of the core temperature Tc can be calculated from the change amount ΔR of the heat resistance of the subcutaneous tissue on the basis of the following Formula (5), and the core temperature Tc can be corrected on the basis of Formula (6).

ΔTc=ΔR/Rr×(Ts−Tu) (5)

Tc=Tc+ΔTc (6)

Incidentally, FIG. 4 illustrates one example of the relation between the blood flow rate vblood and the core temperature depth L. The core temperature depth L is expressed by a function f using the blood flow rate vlood as a variable.

In summary, in the related art, when the blood flow rate vblood increases, the heat resistance Rx is calculated to be larger than the actual one, so the core temperature is overestimated. However, in this embodiment, the value close to the actual core temperature can be obtained by correcting the heat resistance Rx with the blood flow rate.

[Measurement Method of Internal Body Temperature Measurement Device]

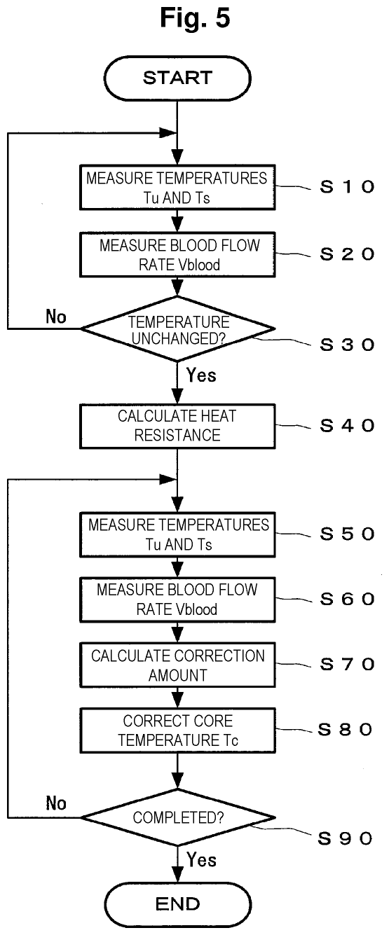

Next, the operation of the internal body temperature measurement device according to this embodiment will be described with reference to FIG. 5.

Incidentally, in the memory 50, the relation L=f(vblood) (see FIG. 4) between the blood flow rate vblood and the apparent core temperature depth L obtained from an experiment or the like is stored in a form of a relational expression f(vblood) or a table.

First, the temperature Tu of the upper surface of the heat resistor 20r and the temperature of the lower surface, that is, the epidermis temperature Ts are measured by using two temperature sensors 20U and 20S of the heat flux sensor 20 (step S10). Further, the blood flow rate of the body surface is measured by using the blood flow sensor 30 (step S20). The operations are repeated a plurality of times to determine whether or not the fluctuation of the upper surface temperature Tu of the heat resistor 20r and the epidermis temperature Ts falls within a predetermined range (step S30). When the fluctuation does not fall within the predetermined range (step S30: No), it is determined that the heat flux is not in a steady state, the procedure returns to step S10, and steps S10 to S30 are repeated.

On the other hand, when the fluctuation of the upper surface temperature Tu of the heat resistor 20r and the epidermis temperature Ts falls within the predetermined range (step S30: Yes), it is determined that the heat flux is in the steady state, and an initial value Tco of the core temperature of the living body 90 is estimated from the magnitude of the heat flux measured by the heat flux sensor 20 (step S40). Specifically, the initial value Tco of the core temperature is calculated from the upper surface temperature Tu of the heat resistor 20r, the epidermis temperature Ts, the heat resistance Rr of the heat resistor 20r, and the heat resistance Rxo of the subcutaneous tissue as a predetermined reference on the basis of Formula (1), for example.

After the initial value Tco of the core temperature Tc is calculated, the upper surface temperature Tu of the heat resistor 20r and the epidermis temperature Ts are measured at a predetermined time interval (step S50), and each time the upper surface temperature Tu and the epidermis temperature Ts are measured, the blood flow rate vblood of the body surface in the vicinity of the heat flux sensor 20 is measured by the blood flow sensor 30 (step S60). Further, the change amount Δvblood of the blood flow rate vblood is calculated, and the change amount ΔR of the heat resistance between the epidermis of the subcutaneous tissue and the temperature region of the core temperature Tc is obtained from the change amount Δvblood of the blood flow rate by using the relation L=f(vblood) (see FIG. 4) between the blood flow rate vblood and the apparent core temperature depth L stored in the memory 50. By using the change amount ΔR of the heat resistance, the correction amount ΔTc of the core temperature Tc is calculated on the basis of Formula (5) (step S70), and the core temperature Tc is corrected on the basis of Formula (6) (step S80).

Further, until an indication of completion is given (step S90: No), the above steps S50 to S80 are repeated, and when the indication of completion is given (step S90: Yes), a series of processing is ended.

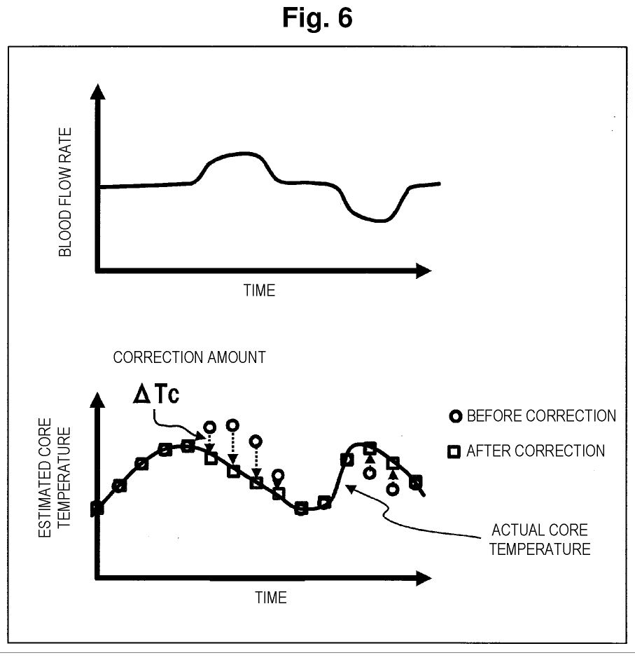

One example of the time-series data of the core temperature Tc obtained by the above correction is illustrated in FIG. 6.

In a case where the change amount ΔR of the heat resistance of the subcutaneous tissue by the change in the blood flow rate is not taken into consideration similarly to the related art, when the blood flow changes, there occurs a measurement error as indicated by a circle. On the other hand, according to the internal body temperature measurement device according to this embodiment, by calculating the change amount ΔTc of the core temperature Tc associated with the change in the blood flow rate and performing correction, the accurate core temperature Tc can be obtained even when the blood flow change occurs. Accordingly, the fluctuation of the core temperature Tc as an internal body rhythm can be grasped more accurately.

### Second Embodiment

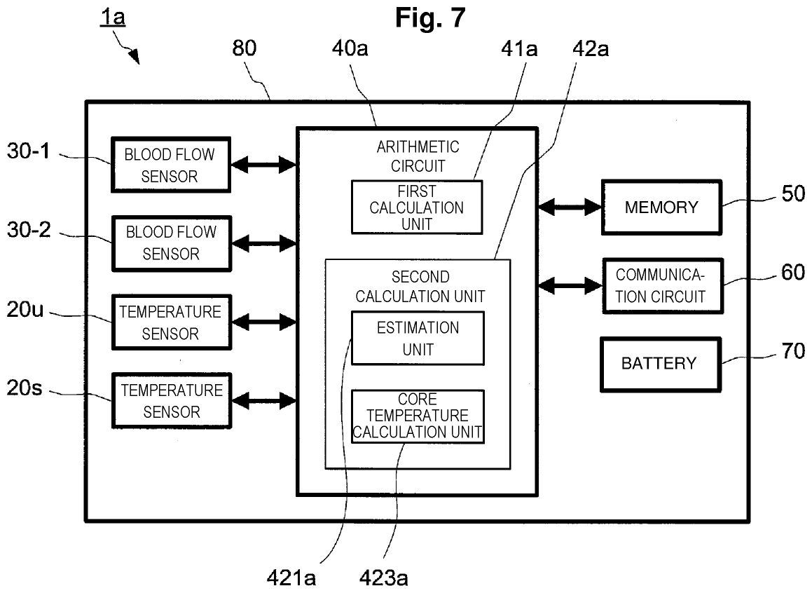

Next, a second embodiment of the present invention and a modification thereof will be described with reference to FIGS. 7 to 9. Incidentally, the same reference symbols are used for the components common to the above-described internal body temperature measurement device 1 according to the first embodiment, and the detailed description thereof will be omitted.

As illustrated in FIG. 7, an internal body temperature measurement device 1a according to the second embodiment of the present invention includes, on the base material 80, the heat flux sensor 20 which includes two temperature sensors 20u and 20s, two blood flow sensors 30-1 and 30-2, an arithmetic circuit 40a, the memory 50, the communication circuit 60 which functions as the I/F circuit with the outside, and the battery 70 which supplies power to the arithmetic circuit 40a, the communication circuit 60, or the like.

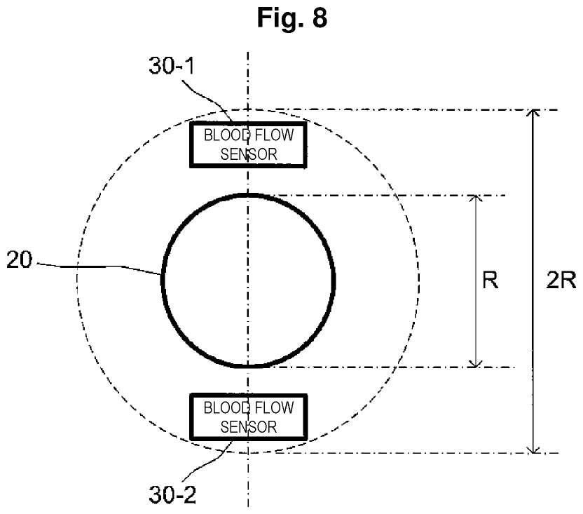

In this embodiment, two blood flow sensors 30-1 and 30-2 are arranged in the vicinity of the heat resistor 20r of the heat flux sensor 20. At this time, when the effect of the region of about twice the diameter R of the heat resistor 20r on the measurement of the core temperature Tc is taken into consideration, as illustrated in FIG. 8, two blood flow sensors 30-1 and 30-2 are installed within the region of twice the diameter R of the heat resistor 20r of the heat flux sensor 20, that is, about the diameter 2R around the heat resistor 20r in plan view.

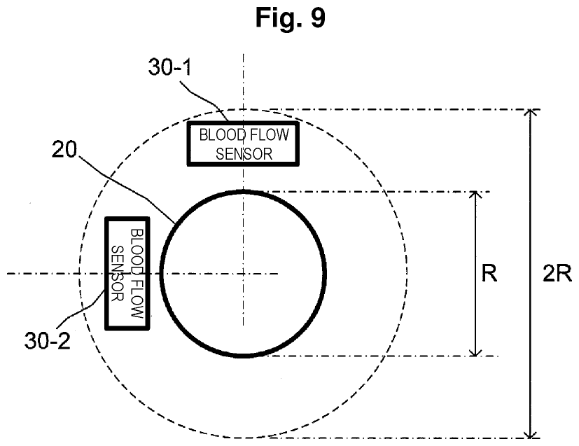

More specifically, as illustrated in FIG. 8, two blood flow sensors 30-1 and 30-2 are arranged at positions of a line object across the heat resistor 20r of the heat flux sensor 20 in plan view. However, the present invention is not limited thereto. For example, as illustrated in FIG. 9, in plan view, two blood flow sensors 30-1 and 30-2 may be arranged to be positioned at two lines passing through the center of the heat resistor 20r of the heat flux sensor 20 and crossing each other at right angles.

On the other hand, in this embodiment, the core temperature Tc is used as the parameter regarding the core temperature of the living body, and the previously prepared relation Tc=g(vblood) between the blood flow rate vblood and the core temperature Tc is stored in the memory 50.

Similarly to the arithmetic circuit 40 of the internal body temperature measurement device 1 according to the first embodiment, the arithmetic circuit 40a includes a first calculation unit 41a which calculates the parameter corresponding to the blood flow rate in the vicinity of the heat flux sensor 20, that is, the correction amount of the value of the core temperature Tc on the basis of the relation stored in the memory 50, and a second calculation unit 42a which calculates the core temperature (Tc+ΔTc) of the living body on the basis of the epidermis temperature Ts measured by the temperature sensor 20S, the magnitude of the heat flux measured by the heat flux sensor 20, and the correction amount ΔTc of the core temperature Tc calculated by the first calculation unit 41a.

Among them, the second calculation unit 42a is configured to include an estimation unit 421a which estimates the core temperature Tc of the living body 90 from the epidermis temperature measured by the temperature sensor 20S and the magnitude of the heat flux measured by the heat flux sensor 20, and a core temperature calculation unit 423a which corrects the core temperature Tc of the living body 90 estimated by the estimation unit 421a on the basis of the correction amount of the value of the parameter calculated by the first calculation unit 41a and calculates the core temperature (Tc+ΔTc) of the living body 90. The above-described arithmetic circuit 40a can be formed from an arithmetic device and a computer program.

Herein, the internal body temperature measurement device is according to this embodiment includes two blood flow sensors 30-1 and 30-2 with respect to one heat flux sensor 20. Thus, the first calculation unit 41a of the arithmetic circuit 40a is configured to obtain the average value of respective blood flow rates measured by the two blood flow sensors 30-1 and 30-2 as a representative value of the blood flow rate and obtain the correction amount ΔTc of the core temperature Tc corresponding to the average value of the blood flow rates by using the relation stored in the memory 50.

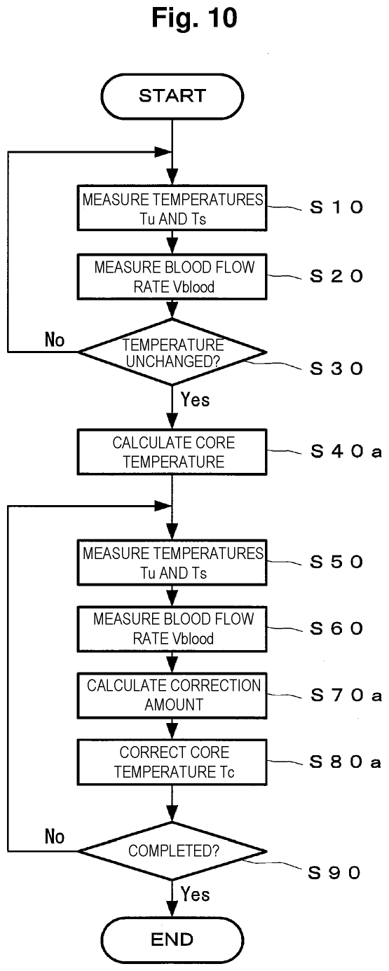

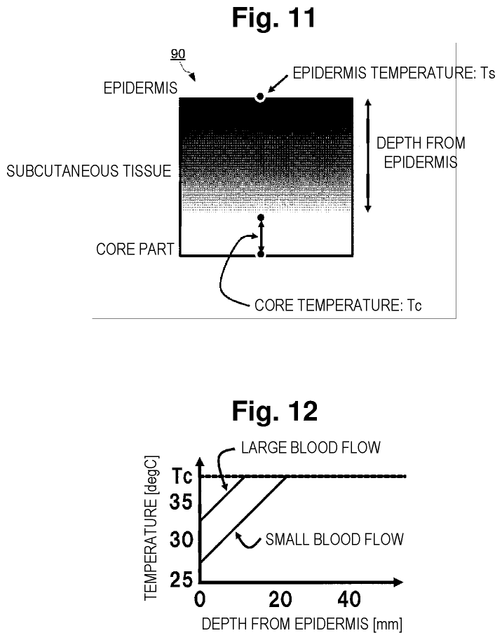

Next, the operation of the internal body temperature measurement device is according to this embodiment will be described with reference to FIG. 10.

First, the temperature Tu of the upper surface of the heat resistor 20r and the temperature of the lower surface, that is, the epidermis temperature Ts are measured by using two temperature sensors 20U and 20S of the heat flux sensor 20 (step S10). Further, the blood flow rate of the body surface is measured by using the blood flow sensor 30 (step S20). The operations are repeated a plurality of times to determine whether or not the fluctuation of the upper surface temperature Tu of the heat resistor 20r and the epidermis temperature Ts falls within the predetermined range (step S30). When the fluctuation does not fall within the predetermined range (step S30: No), it is determined that the heat flux is not in the steady state, the procedure returns to step S10, and steps S10 to S30 are repeated.

On the other hand, when the fluctuation of the upper surface temperature Tu of the heat resistor 20r and the epidermis temperature Ts falls within the predetermined range (step S30: Yes), it is determined that the heat flux is in the steady state, and the initial value Tco of the core temperature of the living body 90 is estimated from the magnitude of the heat flux measured by the heat flux sensor 20 (step S40a). Specifically, the initial value Tco of the core temperature Tc is calculated from the upper surface temperature Tu of the heat resistor 20r, the epidermis temperature Ts, the heat resistance Rr of the heat resistor 20r, and the heat resistance Rxo of the subcutaneous tissue as the predetermined reference on the basis of Formula (1), for example.

After the initial value Tco of the core temperature Tc is calculated, the upper surface temperature Tu of the heat resistor 20r and the epidermis temperature Ts are measured at the predetermined time interval (step S50), and each time the upper surface temperature Tu and the epidermis temperature Ts are measured, the blood flow rate vblood of the body surface in the vicinity of the heat flux sensor 20 is measured by the blood flow sensor 30 (step S60). Further, the change amount Δvblood of the blood flow rate vblood is calculated, the correction amount ΔTc of the core temperature Tc is calculated from the change amount Δvblood of the blood flow rate by using the relation Tc=g(vblood) between the blood flow rate vblood and the core temperature Tc stored in the memory 50 (step S70a), and the core temperature Tc is corrected on the basis of Formula (6) (step S80a).

Further, until the indication of completion is given (step S90: No), the above steps S50 to S80a are repeated, and when the indication of completion is given (step S90: Yes), a series of processing is completed.

According to this embodiment, the correction amount ΔTc of the core temperature Tc associated with the change in the blood flow rate is calculated, and the core temperature Tc calculated on the basis of the epidermis temperature Ts and the magnitude of the heat flux is corrected by the correction amount ΔTc, whereby the accurate core temperature Tc can be obtained even when the blood flow change occurs.

According to this embodiment, the blood flow rate in the vicinity of the heat flux sensor can be measured accurately by using a plurality of blood flow sensors. Thus, the calculation accuracy of the correction amount according to the blood flow rate is improved, and the core temperature can be grasped more accurately.

Incidentally, in this embodiment, two blood flow sensors 30-1 and 30-2 are used. However, of course, three or more blood flow sensors may be used.

Further, as the representative value of respective blood flow rates measured by the plurality of blood flow sensors, the average value of the blood flow rates can be used. However, instead of the average value, a maximum value or a minimum value may be used, and in addition, in the case of using three or more blood flow sensors, a median value or the like may be used.

### REFERENCE SIGNS LIST

1, 1a Internal body temperature measurement device
20 Heat flux sensor
20u, 20s Temperature sensor
20r Heat resistor
30 Blood flow sensor
40 Arithmetic circuit
50 Memory
60 Communication circuit
70 Battery
80 Base material
90 Living body.

1.-8. (canceled)

9. An internal body temperature measurement device comprising:
a temperature sensor which measures an epidermis temperature of a living body;
a heat flux sensor which measures a magnitude of a heat flux discharged from a body surface of the living body;
a blood flow sensor which measures a blood flow rate in a vicinity of the heat flux sensor;
a non-transitory memory which stores a relation between the blood flow rate and a parameter regarding a core temperature of the living body; and
an arithmetic circuit configured to:
obtain a correction amount of a value of the parameter corresponding to the blood flow rate in the vicinity of the heat flux sensor based on the relation; and
calculate the core temperature of the living body from the epidermis temperature, the magnitude of the heat flux, and the correction amount of the value of the parameter.

10. The internal body temperature measurement device according to claim 9, wherein the arithmetic circuit includes:
a first calculator which calculates the correction amount of the value of the parameter corresponding to the blood flow rate in the vicinity of the heat flux sensor based on the relation, and
a second calculator which calculates the core temperature of the living body based on the epidermis temperature, the magnitude of the heat flux, and the correction amount of the value of the parameter calculated by the first calculator.

11. The internal body temperature measurement device according to claim 10, wherein the second calculator includes:
an estimator which estimates a heat resistance between an epidermis and a core part of the living body from the magnitude of the heat flux measured by the heat flux sensor;
a corrector which corrects the heat resistance estimated by estimator based on the correction amount of the value of the parameter; and
a core temperature calculator which calculates the core temperature of the living body based on the epidermis temperature, the magnitude of the heat flux, and the heat resistance obtained by the corrector.

12. The internal body temperature measurement device according to claim 10, wherein the second calculator includes:
an estimator which estimates a depth from an epidermis to a core part of the living body from the magnitude of the heat flux measured by the heat flux sensor,
a corrector which corrects the depth from the epidermis to the core part of the living body estimated by the estimator based on the correction amount of the value of the parameter; and
a core temperature calculator which calculates the core temperature of the living body based on the epidermis temperature, the magnitude of the heat flux, and the depth from the epidermis to the core part of the living body obtained by the corrector.

13. The internal body temperature measurement device according to claim 10, wherein the second calculator includes:
an estimator which estimates an estimated core temperature of the living body from the epidermis temperature and the magnitude of the heat flux; and
a core temperature calculator which calculates the core temperature of the living body by correcting the estimated core temperature of the living body estimated by the estimator based on the correction amount of the value of the parameter calculated by the first calculator.

14. The internal body temperature measurement device according to claim 9, wherein the parameter is a depth from an epidermis of the living body to a core part of the living body, a heat resistance between the epidermis and the core part, or the core temperature.

15. The internal body temperature measurement device according to claim 9, further comprising two blood flow sensors, wherein the arithmetic circuit:
obtains a representative value of respective blood flow rates measured by the two blood flow sensors; and
obtains the correction amount of the value of the parameter corresponding to the blood flow rate in the vicinity of the heat flux sensor based on the representative value of the respective blood flow rates and the relation.

16. An internal body temperature measurement method comprising:
measuring an epidermis temperature of a living body and a magnitude of a heat flux discharged from a body surface of the living body;
measuring a blood flow rate of the body surface; and
obtaining, based on a relation between a blood flow rate in a vicinity of a heat flux sensor and a parameter regarding a core temperature of the living body, a correction amount of a value of the parameter corresponding to the blood flow rate in the vicinity of the heat flux sensor; and
calculating the core temperature of the living body from the epidermis temperature, the magnitude of the heat flux, and the correction amount of the value of the parameter.

17. The method according to claim 16, wherein the relation between the blood flow rate in the vicinity of the heat flux sensor and the parameter regarding the core temperature of the living body is pre-stored in a memory.
